(19) United States
(12) Patent Application Publication
(10) Pub. No.: US 2005/0107628 A1
(43) Pub. Date: May 19, 2005

(54) Modified method for producing surfactant alcohols and surfactant alcohol ethers, the products of said method and use of the same

(75) Inventors: Michael Roper, Wachenheim (DE); Jurgen Stephan, Mannheim (DE); Gotz-Peter Schindler, Mannheim (DE); Jurgen Tropsch, Romerberg (DE); Thomas Heidemann, Viernheim (DE); Martina Prinz, Dossenheim (DE); Soeren Zimdahl, Schriesheim (DE)

Correspondence Address: NOVAK DRUCE DELUCA & QUIGG, LLP, 1300 EYE STREET NW, SUITE 400 EAST, WASHINGTON, DC, 20036, US

(73) Assignee: BASF Aktiengesellschaft, Ludwigshafen (DE)

(21) Appl. No.: 10/503,213

(22) PCT Filed: Feb. 19, 2003
(86) PCT No.: PCT/EP03/01668

(30) Foreign Application Priority Data
Feb. 19, 2002 (DE) 102 06 845.3

(51) Int. Cl. C07C 9/00; C07H 13/02; C07F 9/02; C07C 27/12

(52) U.S. Cl. 558/20; 585/16; 558/104; 568/671; 568/910; 536/119

(57) ABSTRACT

A process for preparing surfactant alcohols and surfactant alcohol ethers by derivatization of olefins having about 10 to 20 carbon atoms or of mixtures of such olefins and optional subsequent alkoxylation is described. As features, the process involves
(a) subjecting a C₄-olefin mixture with a 1-butene/2-butene ratio of ≧1.2 to a metathesis, (b) separating off olefins having 5 to 8 carbon atoms from the metathesis mixture, (c) subjecting the separated-off olefins individually or in a mixture to a dimerization to give olefin mixtures having 10 to 16 carbon atoms, (d) subjecting the resulting olefin mixture, optionally after a fractionation, to derivatization to give a mixture of surfactant alcohols and (e) optionally alkoxylating the surfactant alcohols. The olefin mixture obtained in the dimerization comprises a high content of branched components and less than 10% by weight of compounds which contain a vinylidene group. Also described is the use of surfactant alcohols and surfactant alcohol ethers to give surfactans by glycosidation or polyglycosidation, sulfation or phosphation.

The present invention relates to a process for preparing surfactant alcohols and surfactant alcohol ethers which are very highly suitable, inter alia, as surfactants or for preparing surfactants. In this process, olefins or olefin mixtures are prepared starting from C₄-olefin streams by a metathesis reaction and are dimerized to give an olefin mixture having 10 to 16 carbon atoms which comprises less than 10% by weight of compounds which have a vinylidene group, the olefins are then derivatized to give surfactant alcohols and these are optionally alkoxylated. The C₄-olefins are prepared as described in the present application.

The invention further relates to the use of the surfactant alcohols and surfactant alcohol ethers for the preparation of surfactants by glycosidation or polyglycosidation, sulfation or phosphation.

Fatty alcohols having chain lengths of from C₈ to C₁₈ are used for the preparation of nonionic surfactants. They are reacted with alkylene oxides to give the corresponding fatty alcohol ethoxylates (chapter 2.3 in: Kosswig/Stache, “Die Tenside” [Surfactants], Carl Hanser Verlag, Munich Vienna (1993)). Here, the chain length of the fatty alcohol influences various surfactant properties, such as, for example, wetting ability, foam formation, fat-release capacity, cleaning power.

Fatty alcohols having chain lengths of from C₈ to C₁₈ can also be used for preparing anionic surfactants, such as alkyl phosphates and alkyl ether phosphates. Instead of phosphates, it is also possible to prepare the corresponding sulfates (chapter 2.2 in: Kosswig/Stache, “Die Tenside” [Surfactants], Carl Hanser Verlag, Munich Vienna (1993)).

Such fatty alcohols are obtainable from natural sources, e.g. from fats and oils, or else in a synthetic manner by construction from building blocks having a lower number of carbon atoms. One variant here is the dimerization of an olefin to give a product having twice the number of carbon atoms and its functionalization to give an alcohol.

For the dimerization of olefins, a number of processes are known. For example, the reaction can be carried out over a heterogeneous cobalt oxide/carbon catalyst (DE-A-1 468 334), in the presence of acids, such as sulfuric acid or phosphoric acid (FR 964 922), with an alkylaluminum catalyst (WO 97/16398), or with a dissolved nickel complex catalyst (U.S. Pat. No. 4,069,273). According to the details in U.S. Pat. No. 4,069,273, the use of these nickel complex catalysts—the complexing agent used being 1,5-cyclooctadiene or 1,1,1,5,5,5-hexafluoropentane-2,4-dione—gives highly linear olefins with a high proportion of dimerization products.

The functionalization of the olefins to give alcohols with build-up of the carbon backbone by one carbon atom expediently takes place via the hydroformylation reaction, which produces a mixture of aldehydes and alcohols, which can subsequently be hydrogenated to give alcohols. Approximately 7 million tonnes of products are produced annually worldwide using the hydroformylation of olefins. An overview of catalysts and reaction conditions of the hydroformylation process are given, for example, by Beller et al. in Journal of Molecular Catalysis, A104 (1995), 17-85 and also Ullmanns Encyclopedia of Industrial Chemistry, vol. A5 (1986), page 217 et seq., page 333, and the relevant literature references.

From WO 98/23566 it is known that sulfates, alkoxylates, alkoxysulfates and carboxylates of a mixture of branched alkanols (oxo alcohols) exhibit good surface activity in cold water and have good biodegradability. The alkanols in the mixture used have a chain length of more than 8 carbon atoms, having on average 0.7 to 3 branches. The alkanol mixture can be prepared, for example, by hydroformylation, from mixtures of branched olefins which for their part can be obtained either by skeletal isomerization or by dimerization of internal, linear olefins.

A stated advantage of the process is that no C₃- or C₄-olefin stream is used for preparing the dimerization feed. It follows from this that, according to the current prior art, the olefins subjected to dimerization must have been prepared from ethylene (e.g. SHOP process). Since ethylene is a relatively expensive starting material for surfactant preparation, ethylene-based processes have an economic disadvantage compared with processes which start from C₃- and/or C₄-olefin streams.

A further disadvantage of this known process is the use of mixtures of internal olefins required for the production of branched surfactant oxo alcohols; these are only accessible by isomerization of alpha-olefins. Such processes always lead to isomer mixtures which, due to the differing physical and chemical data of the components, are more difficult to handle in terms of processing than pure substances. Furthermore, the additional process step of isomerization is required, which gives the process a further disadvantage.

The structure of the components of the oxo alkanol mixture depends on the type of olefin mixture which has been subjected to the hydroformylation. Olefin mixtures which have been obtained by skeletal isomerization from alpha-olefin mixtures lead to alkanols which are branched predominantly at the ends of the main chain, i.e. in positions 2 and 3, calculated from the end of the chain in each case (page 56, last paragraph). According to the process disclosed in this publication, olefin mixtures which have been obtained by dimerization of olefins of shorter chain length produce oxo alcohols whose branches are more in the middle of the main chain, and, as table IV on page 68 shows, very predominantly on C4 and further removed carbon atoms, relative to the hydroxylcarbon atom. By contrast, fewer than 25% of the branches are found at the C2 and C3 positions, relative to the hydroxylcarbon atom (pages 28/29 of this document).

The surface-active end products are obtained from the alkanol mixtures either by oxidation of the —CH₂OH group to the carboxyl group, or by sulfation of the alkanols or their alkoxylates.

Similar processes for preparing surfactants are described in the PCT patent application WO 97/38957 and in EP-A-787 704. Also in the process described therein, an alpha-olefin is dimerized to give a mixture of predominantly vinylidene-branched olefin dimers (WO 97/38957):

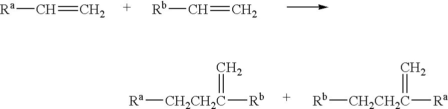

The vinylidene compounds are then double-bond-isomerized such that the double bond migrates from the end of the chain further into the center, and are then subjected to hydroformylation to give an oxo alcohol mixture. The latter is then further reacted, e.g. by sulfation, to give surfactants. A serious disadvantage of this process is that it starts from alpha-olefins. Alpha-olefins are obtained, for example, by transition-metal-catalyzed oligomerization of ethylene, Ziegler build-up reaction, wax cracking or Fischer-Tropsch process and are therefore relatively expensive starting materials for the preparation of surfactants. A further considerable disadvantage of this known surfactant preparation process is that a skeletal isomerization must be inserted in the process between the dimerization of the alpha-olefins and the hydroformylation of the dimerization product if predominantly branched products are desired. Due to the use of a starting material which is relatively expensive for surfactant production, and the need to insert an additional process step, the isomerization, this known process is at a considerable economic disadvantage.

WO 00/39058 states that, for the preparation of branched olefins and alcohols (oxo alcohols), which can be further processed to give highly effective surfactants (referred to below as “surfactant alcohols”), it is not necessary to be dependent on either alpha-olefins or on olefins which have been produced mainly from ethylene, but that it is possible to start from inexpensive C₄-olefin streams, and that, moreover, the isomerization step can be avoided by working in accordance with the process described in this document.

A process for preparing surfactant alcohols and surfactant alcohol ethers by derivatization of olefins having about 10 to 20 carbon atoms or of mixtures of such olefins and optional subsequent alkoxylation is disclosed, which comprises
a) subjecting a C₄-olefin mixture to metathesis
b) separating off olefins having 5 to 8 carbon atoms from the metathesis mixture,
c) subjecting the separated-off olefins individually or in a mixture to a dimerization to give olefin mixtures having 10 to 16 carbon atoms,
d) subjecting the resulting olefin mixture, optionally after a fractionation, to derivatization to give a mixture of surfactant alcohols and
e) optionally alkoxylating the surfactant alcohols.

The C₄-olefin streams used in the process are mixtures which consist essentially, preferably of more than 80 to 85% by volume, in particular of more than 98% by volume, of 1-butene and 2-butene, and to a lesser extent—usually not more than 15 to 20% by volume—of n-butane and isobutane as well as traces of polyunsaturated C₄-hydrocarbons, and C₅-hydrocarbons. These hydrocarbon mixtures, also referred to in the specialist jargon as “raffinate II”, are by-produced in the cracking of high molecular weight hydrocarbons, e.g. of crude oil. The low molecular weight olefins which form in this process, ethene and propene, are valuable raw materials for preparing polyethylene and polypropylene, and the hydrocarbon fractions above C₆ are used as fuels in combustion engines and for heating purposes.

Raffinate II, in particular its C₄-olefins, could not be further processed to an adequate degree to give valuable surfactant alcohols before the teaching of WO 00/39058 became known. The process then opened up a way, which was very favorable with regard to the process, of processing the C₄-olefin streams obtained to give valuable surfactant alcohols, from which nonionic or anionic surfactants could then be prepared by various processes known per se.

The teaching of WO 00/39058 is suitable in the strict sense only for the processing of “raffinate II”. Raffinate II is obtained by cleaving high molecular weight hydrocarbons, for example naphtha or gas oil. However, C₄ mixtures which are obtained from other sources are also suitable in principle as feed product for the metathesis reaction followed by the further reaction steps described in WO 00/39058. For example, the dehydrogenation of butanes is a suitable C₄-olefin source. This process, known per se, is described, for example, in U.S. Pat. No. 4,788,371, WO 94/29021, U.S. Pat. No. 5,733,518, EP-A 0 383 534, WO 96/33151, WO 96/33150 and DE-A 100 47 642, and all of the abovementioned documents in principle disclose suitable dehydrogenation processes which can, where appropriate, be adapted to the respective requirements.

Another way of obtaining C₄ feed olefins is the “MTO” (methanol to olefin) process. In this process, methanol is converted to olefins over suitable zeolitic catalysts. Optionally, the methanol is prepared from C1 hydrocarbons. The MTO process is described, for example, in Weissermel, Arpe, Industrielle Organische Chemie [Industrial Organic Chemistry], 5th edition 1998, pages 36 to 38.

C₄-Olefin mixtures suitable as starting material can also be obtained by metathesis of propene (Phillips Triolefin Process), by the Fischer-Tropsch process, or the dimerization of ethylene, and also by the partial hydrogenation of butadiene.

If surfactant alcohols are produced in the manner described in WO 00/39058 from the very diverse C₄-olefin mixtures (which in principle can originate from any of the abovementioned processes, in particular the olefin mixture is raffinate II), then the challenge of carrying out the process differently presents itself.

Thus, WO 00/39058 gives no information as to what composition the raffinate II used should have. In particular, there is not even any information with regard to the required 1-butene/2-butene ratio in such a raffinate. In the example, which describes the metathesis of raffinate II, this has a 1-butene/2-butene ratio of 1.06. From this, it can be concluded that in the process according to WO 00/39058, the metathesis stage a) with a raffinate II can be carried out in any desired composition prepared in the customary industrial production processes.

With regard to carrying out the metathesis step a) of WO 00/39058 or other starting material mixtures to be used, prior art exists which is reproduced below.

EP-A-0 742 195 relates to a process for converting C₄ or C₅ cuts into ether and propylene. Starting from C₄ cuts, diolefins and acetylenic impurities present are firstly selectively hydrogenated, the hydrogenation being combined with an isomerization of 1-butene to 2-butene. The yield of 2-butenes should be maximized. The ratio of 2-butene to 1-butene after the hydrogenation is about 9:1. It is followed by an etherification of the isoolefins present, the ethers being separated off from the C₄ cut. Oxygenate impurities are then separated off. The exit stream obtained, which comprises predominantly 2-butene in addition to alkanes, is then reacted with ethylene in the presence of a metathesis catalyst in order to obtain a reaction exit stream which comprises propylene as product. The metathesis is carried out in the presence of a catalyst which comprises rhenium oxide on a support.

DE-A-198 13 720 relates to a process for preparing propene from a C₄ stream. Here, butadiene and isobutene are firstly removed from the C₄ stream. Oxygenate impurities are then separated off, and a two-stage metathesis of the butenes is carried out. Firstly, 1-butene and 2-butene are converted to propylene and 2-pentene. Then, the resulting 2-pentene is reacted further with metered-in ethylene to give propylene and 1-butene.

DE-A-199 32 060 relates to a process for preparing C₅-/C₆-olefins by reacting a starting stream which comprises 1-butene, 2-butene and isobutene, in a metathesis to give a mixture of C₂₋₆-olefins. Here, propene, in particular, is obtained from butenes. Additionally, hexene and methylpentene are discharged as products. In the metathesis, no ethene is metered in. Optionally, ethene formed in the metathesis is returned to the reactor.

DE-A 100 13 537 discloses a process for preparing propene and hexene from an olefinic C₄-hydrocarbon-containing raffinate II starting stream in which
a) in the presence of a metathesis catalyst which comprises at least one compound of a metal of subgroup VI.b, VII.b or VIII of the Periodic Table of the Elements, a metathesis reaction is carried out in the course of which butenes present in the starting stream are reacted with ethene to give a mixture containing ethene, propene, butenes, 2-pentene, 3-hexene and butanes, 0.05 to 0.6 molar equivalent of ethene being used, based on the butenes,
b) the exit stream obtained in this way is firstly separated by distillation into a low-boiling fraction A containing C₂-C₃-olefins, and into a high-boiling fraction containing C₄-C₆-olefins and butanes,
c) the low-boiling fraction A obtained from b) is then separated by distillation into a fraction containing ethene and a fraction containing propene, where the fraction containing ethene is returned to process step a), and the fraction containing propene is discharged as product,
d) the high-boiling fraction obtained from b) is then separated by distillation into a low-boiling fraction B containing butenes and butanes, a mid-boiling fraction C containing pentene, and into a high-boiling fraction D containing hexene,
e) where the fractions B and C are returned completely or partially to process step a), and the fraction D is removed from the system as product.

The world market prices of ethene and propene have been subjected to changes. For this reason, the preparation costs for the pentene and hexene olefin fractions which are obtainable by the process as in WO 00/39058 vary to a relatively high degree. It is clear from the statements made above that the resulting spectrum of products of value can be adapted to the price situation by varying the feed amounts of ethylene, depending on the price difference between ethene and propene. There is, however, hitherto no process which allows a product spectrum to be obtained without the addition or with the addition of only small amounts of ethylene which produces a large amount of olefins which are suitable for preparing surfactant alcohols which satisfy modern requirements with regard to biodegradability and suitability as detergent raw material, but at the same time produces by-products which can be utilized economically.

It is an object of the present invention to provide a process for producing surfactant alcohols in accordance with the teaching of WO 00/39058, which allows a favorable product spectrum both with regard to said olefins and also with regard to distribution of the by-products to be obtained in the production of 2-pentene and in particular 3-hexene by metathesis and subsequent dimerization without having to have recourse to, ideally even being able to dispense with, the addition of relatively large amounts of ethylene. In this way, the addition of value to the surfactant alcohol synthesis should be improved, particularly as a result of obtaining a valuable by-product spectrum.

We have found that this object is achieved by a process for preparing surfactant alcohols and surfactant alcohol ethers by derivatization of olefins having about 10 to 20 carbon atoms or of mixtures of such olefins and optional subsequent alkoxylation, which comprises
a) subjecting a C₄-olefin mixture with a 1-butene/2-butene ratio of ≧1.2 to a metathesis,
b) separating off olefins having 5 to 8 carbon atoms from the metathesis mixture,
c) subjecting the separated-off olefins individually or in a mixture to a dimerization to give olefin mixtures having 10 to 16 carbon atoms,
d) subjecting the resulting olefin mixture, optionally after a fractionation, to derivatization to give a mixture of surfactant alcohols and
e) optionally alkoxylating the surfactant alcohols.

The process according to the invention is successful in many cases, particularly when the 1-butene/2-butene ratio is close to the ideal value of about 2, without the addition of ethylene. This means that small amounts of ethylene, generally <20 mol %, based on n-butenes, are added. Particularly in the case of 1-butene/2-butene ratios close to the lower limit of 1.2, it is still possible thereby to obtain a favorable spectrum of products of value (formation of propene).

In order to illustrate the process according to the invention in more detail in a plurality of variations, the reaction which takes place in the metathesis reactor in stage a) is divided into three important individual reactions:
1. Cross-Metathesis of 1-butene with 2-butene 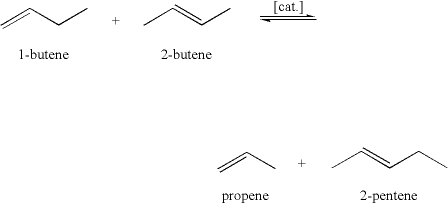
2. Self-Metathesis of 1-butene 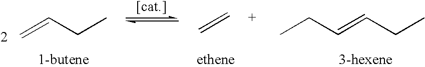
3. Ethenolysis of 2-butene

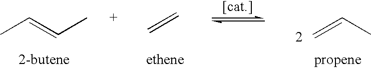

Depending on the respective demand for the target products propene and 3-hexene (the designation 3-hexene includes, inter alia, any isomers formed), the external mass balance of the process can be influenced in a targeted manner by shifting the equilibrium by recirculation of certain substreams. Thus, for example, the yield of 3-hexene is increased by suppressing the cross-metathesis of 1-butene with 2-butene by recirculating 2-pentene to the metathesis step, so that no or as little as possible 1-butene is consumed. During the self-metathesis of 1-butene to 3-hexene, which then proceeds in preference, ethene is additionally formed, which reacts in a subsequent reaction with 2-butene to give the product of value propene.

The advantage of a high 1-butene/2-butene ratio according to the invention becomes clear in the following considerations.

For a molar 1-butene/2-butene ratio of 2:1 (according to the invention), the products of value 3-hexene and propene are formed in equal amounts in the above process with recirculation of 2-pentene and ethylene. To produce 100 parts by weight of 3-hexene, 200 parts by weight of (1-butene+2-butene) are required, and 100 parts by weight of propene are formed. If the 1-butene excess is lower, less 3-hexene is formed and 2-butene remains, which can be converted into propene with ethylene additionally fed in from outside. Thus, when a 1-butene/2-butene ratio of 1:1 (not in accordance with the invention) is used, 267 parts by weight of (1-butene+2-butene) are needed for producing the same amount of product of value 3-hexene. The stream of the 2-pentene to be returned to the process increases greatly and 67 parts by weight of 2-butene remain unreacted. These can only be converted into the product of value by additionally introducing ethylene. To convert the mentioned 67 parts by weight of 2-butene, 33 parts by weight of ethylene would be required, whereupon 100 parts by weight of propene would then additionally form.

A further surprising advantage of the process according to the invention is that the catalyst cycle times during the metathesis reaction are considerably extended compared to a process which is unable to dispense with the addition of, in particular relatively large, amounts of ethylene during the metathesis. This advantage of the process according to the invention is particularly marked when only small amounts or no ethylene at all is/are added. The catalyst cycle times for the process according to the invention are significantly extended compared with the processes of the prior art. In some cases, up to 100% longer cycle times could be observed.

Olefin mixtures which contain 1-butene and 2-butene and optionally isobutene and can be used in the process according to the invention are obtained, inter alia, as C₄ fraction in diverse cracking processes, such as steamcracking or FCC cracking. Alternatively, it is possible to use butene mixtures as are produced in the dehydrogenation of butanes or by dimerization of ethene. In addition, LPG, LNG or MTO streams can be used. Butanes present in the C₄ fraction behave inertly.

Dienes, alkynes or enynes are removed to unharmful residual amounts prior to the metathesis step according to the invention using customary processes, such as extraction or selective hydrogenation.

The butene content of the C₄ fraction used in the process is 1 to 100% by weight, preferably 60 to 90% by weight. The butene content refers here to 1-butene, 2-butene and isobutene.

In one embodiment of the present invention, a C₄ fraction is thus used as is produced during steam or FCC cracking or during the dehydrogenation of butane. Alternatively a C₄-olefin mixture can be prepared from LPG, LNG or MTO streams.

In this connection, if an LPG stream is used, the olefins are preferably obtained by dehydrogenation of the C₄ fraction of the LPG stream and subsequent removal of any dienes formed, alkynes and enynes, where the C₄ fraction of the LPG stream is separated off before or after the dehydrogenation or removal of dienes, alkynes and enynes from the LPG stream.

If a LNG stream is used, this is preferably converted into the C₄-olefin mixture via a MTO process.

It has been found according to the invention that by using C₄-olefin mixtures with a 1-butene/2-butene ratio of ≧1.2 in the metathesis, products are obtained which can be dimerized to give slightly branched C₁₀₋₁₂-olefin mixtures. These mixtures can be used advantageously in the hydroformylation, giving alcohols which, following ethoxylation and optional further processing such as sulfation, phosphation or glycosidation, produce surfactants which have excellent properties, in particular with regard to sensitivity toward hardness-forming ions, solubility and viscosity of the surfactants and their washing properties.

Moreover, the present process is extremely cost-effective since the product streams can be arranged so that no by-products are formed. Starting from a C₄ stream, the metathesis according to the invention generally prepares linear, internal olefins which are then converted into branched olefins via the dimerization step.

The preparation of a C₄-olefin mixture from LPG, LNG or MTO streams is described below. LPG here means liquefied petroleum gas (liquid gases). Such liquid gases are defined, for example, in DIN 51 622. They generally comprise the hydrocarbons propane, propene, butane, butene and mixtures thereof which are produced in oil refineries as by-products during the distillation and cracking of petroleum, and in the preparation of natural gas during the separation of gasoline. LNG means liquefied natural gas (natural gas). Natural gas consists primarily of saturated hydrocarbons which have varying compositions depending on their origin and are generally divided into three groups. Natural gas from pure natural gas deposits consists of methane and a small amount of ethane. Natural gas from crude oil deposits additionally comprises relatively large amounts of higher molecular weight hydrocarbons, such as ethane, propane, isobutane, butane, hexane, heptane and by-products. Natural gas from condensate and distillate deposits comprises not only methane and ethane, but also higher-boiling components having more than 7 carbon atoms to a considerable degree. For a more detailed description of liquid gases and natural gas, reference may be made to the corresponding keywords in Römpp, Chemielexikon, 9th edition.

The LPG and LNG used as feedstock includes, in particular, “field butanes”, the term used for the C4 fraction of the “moist” fractions of natural gas and of crude oil accompanying gases, which are separated off from the gases in liquid form by drying and cooling to about −30° C. Low-temperature or pressure distillation thereof gives the field butanes, the composition of which varies depending on the deposit, but which generally comprise about 30% of isobutane and about 65% of n-butane.

The C₄-olefin mixtures used for the preparation according to the invention of surfactant alcohols and which are derived from LPG or LNG streams can be obtained in a suitable manner by separating off the C₄ fraction and dehydrogenation, and feed purification. Possible work-up sequences for LPG and LNG streams are dehydrogenation, subsequent removal or partial hydrogenation of the dienes, alkynes and enynes and subsequent isolation of the C₄-olefins. Alternatively, the dehydrogenation can firstly be followed by isolation of the C₄-olefins, followed by removal or partial hydrogenation of the dienes, alkynes and enynes, and optionally further by-products. It is also possible to carry out the sequence of isolation of the C₄-olefins, dehydrogenation, removal or partial hydrogenation. Suitable processes for the dehydrogenation of hydrocarbons are described, for example, in DE-A-100 47 642. The dehydrogenation can be carried out, for example, in one or more reaction zones under heterogeneous catalysis, where at least some of the required heat of dehydrogenation is generated in at least one reaction zone by burning hydrogen, the hydrocarbon(s) and/or carbon in the presence of an oxygen-containing gas directly within their reaction mixture. The reaction gas mixture, which comprises the hydrocarbon(s) to be dehydrogenated, is brought into contact with a Lewis-acidic dehydrogenation catalyst which does not have Brönsted acidity. Suitable catalyst systems are Pt/Sn/Cs/K/La on oxidic supports such as ZrO₂, SiO₂, ZrO₂/SiO₂, ZrO₂/SiO₂/Al₂O₃, Al₂O₃, Mg(Al)O.

Suitable mixed oxides of the support are obtained by successive or common precipitation of soluble precursor substances.

In addition, for the dehydrogenation of alkanes, reference can be made to U.S. Pat. No. 4,788,371, WO 94/29021, U.S. Pat. No. 5,733,518, EP-A-0 838 534, WO 96/33151 or WO 96/33150.

The LNG stream can, for example, be converted into the C₄-olefin mixture via an MTO process. MTO stands here for methanol-to-olefin. It is related to the MTG process (methanol-to-gasoline). It is a process for the dehydration of methanol over a suitable catalyst, giving an olefinic hydrocarbon mixture. Depending on the C₁ feedstream, a methanol synthesis can be connected upstream in the MTO process. C₁ feedstreams can thereby be converted, via methanol and the MTO process, into olefin mixtures from which the C₄ olefins can be separated off by suitable processes. Removal can take place, for example, by distillation. For the MTO process, reference may be made to Weissermel, Arpe, Industrielle organische Chemie, 5th edition 1998, VCH-Verlagsgesellschaft, Weinheim, pp. 36-38.

The methanol-to-olefin process is also described in P. J. Jackson, N. White, Technologies for the Conversion of Natural Gas, Austr. Inst. Energy Conference 1985.

The C₄-olefin mixtures can also be prepared by metathesis of propene (Phillipps triolefin process). The metathesis here can be carried out as described in the present application. In addition, the C₄-olefin mixtures can be obtained by a Fischer-Tropsch process (gas to liquid) or by ethene dimerization. Suitable processes are described in the book already cited by Weissermel and Arpe on p. 23 ff and 74 ff.

Further suitable processes for the preparation of a C₄-olefin mixture are the olefin mixtures obtained by FCC and steamcracking or by partial hydrogenation of butadiene. In this respect, reference has already been made to DE-A-100 39 995.

Where a crude C₄ cut from steamcrackers or FCC crackers is used, the following partial steps can be carried out to prepare C₅/C₆-olefins and propene:
(1) removal of butadiene and acetylenic compounds by optional extraction of butadiene with a butadiene-selective solvent and subsequently or alternatively selective hydrogenation of butadienes and acetylenic impurities present in crude C₄ cut to give a reaction exit stream which comprises n-butenes and isobutene and essentially no butadienes and acetylenic compounds,
(2) removal of isobutene by reaction of the reaction exit stream obtained in the preceding stage with an alcohol in the presence of an acidic catalyst to give an ether, removal of the ether and of the alcohol, which can be carried out simultaneously or after the etherification, to give a reaction exit stream which comprises n-butenes and optionally oxygenate impurities, it being possible to discharge the ether formed or back-cleave it to obtain pure isobutene, and to follow the etherification step by a distillation step for removal of isobutene, where introduced C₃-, i-C₄- and C₅-hydrocarbons can optionally also be removed by distillation in the course of the work-up of the ether, or oligomerization or polymerization of isobutene from the reaction exit stream obtained in the preceding stage in the presence of an acidic catalyst whose acid strength is suitable for the selective removal of isobutene as oligo- or polyisobutene, to give a stream containing 0 to 15% of residual isobutene,
(3) removal of the oxygenate impurities from the exit stream of the preceding steps over appropriately selected absorber materials,
(4) metathesis reaction of the resulting raffinate II stream as described.

Optionally, butanes present are separated off by the customary measures known to the person skilled in the art, for example by distillation, selective extraction or extractive distillation. In particular, isobutane can be separated off by distillation. For selective extraction and extractive distillation, the customary solvents known to the person skilled in the art are used, for example N-methylpyrrolidone (NMP).

Preferably, the partial step of selective hydrogenation of butadiene and acetylenic impurities present in the crude C₄ cut is carried out in two stages by bringing the crude C₄ cut into contact, in the liquid phase, with a catalyst which comprises a metal chosen from the group consisting of nickel, palladium and platinum, on a support, preferably palladium on aluminum oxide, at a temperature of from 20 to 200° C., a pressure of from 1 to 50 bar, a liquid hourly space velocity of from 0.5 to 30 m³ of fresh feed per m³ of catalyst per hour and a ratio of recycle to feedstream of from 0 to 30 with a molar ratio of hydrogen to diolefins of from 0.5 to 50, to give a reaction exit stream in which, apart from isobutene, the n-butenes, 1-butene and 2-butene are present in a molar ratio of ≧1.2, preferably ≧1.4, more preferably ≧1.8 and in particular about 2. Preferably, substantially no diolefins and acetylenic compounds are present here.

For a maximum hexene exit stream, 1-butene is preferably present in excess in the abovementioned 1-butene/2-butene ratios of ≧1.2, preferably ≧1.4, in particular ≧1.8. The optimum is at the mentioned 1-butene/2-butene ratio of about 2. It is of course also possible to use butene mixtures in which the 1-butene/2-butene molar ratio has yet higher, arbitrary values. However, the best product distribution results with a ratio of 2.

Preferably, the partial step of butadiene extraction from crude C₄ cut is carried out with a butadiene-selective solvent chosen from the class of polar-aprotic solvents, such as acetone, furfural, acetonitrile, dimethylacetamide, dimethylformamide and N-methylpyrrolidone in order to obtain a reaction exit stream in which the n-butenes 1-butene and 2-butene are present in a molar ratio of 2:1 to 1:10, preferably from 2:1 to 1:2.

The partial step of isobutene etherification is preferably carried out in a three-stage reactor cascade using methanol or isobutanol, preferably isobutanol, in the presence of an acidic ion exchanger, in which flooded fixed-bed catalysts are flowed through from top to bottom, the reactor inlet temperature being 0 to 60° C., preferably 10 to 50° C., the outlet temperature being 25 to 85° C., preferably 35 to 75° C., the pressure being 2 to 50 bar, preferably 3 to 20 bar, and the ratio of isobutanol to isobutene being 0.8 to 2.0, preferably 1.0 to 1.5, and the overall conversion corresponding to the equilibrium conversion.

Preferably, the partial step of isobutene removal is carried out by oligomerization or polymerization of isobutene starting from the reaction exit stream obtained by the above-described stages of butadiene extraction and/or selective hydrogenation in the presence of a catalyst which is chosen from the class of homogeneous and heterogeneous Brönsted acids, preferably from heterogeneous catalysts which comprise an oxide of a metal of sub-group VI.b of the Periodic Table of the Elements even for an acidic inorganic support, particularly preferably WO₃/TiO₂, in order to produce a stream which has an isobutene residual content of less than 15%.

Selective Hydrogenation of Crude C₄ Cut

Alkynes, alkynenes and alkadienes are undesired substances in many industrial syntheses owing to their tendency to polymerize or their pronounced tendency to form complexes with transition metals. They sometimes have a very strong adverse effect on the catalysts used in these reactions.

The C₄ stream of a steamcracker contains a high proportion of polyunsaturated compounds such as 1,3-butadiene, 1-butyne (ethylacetylene) and butenyne (vinylacetylene). Depending on the downstream processing present, the polyunsaturated compounds are either extracted (butadiene extraction) or are selectively hydrogenated. In the former case, the residual content of polyunsaturated compounds is typically 0.05 to 0.3% by weight, and in the latter case is typically 0.1 to 4.0% by weight. Since the residual amounts of polyunsaturated compounds likewise interfere in the further processing, a further concentration by selective hydrogenation to values <10 ppm is necessary. In order to obtain the highest possible product of value proportion of butenes, over-hydrogenation to butanes must be kept as low as possible.

Suitable hydrogenation catalysts are described in:

J. P. Boitiaux, J. Cosyns, M. Derrien and G. Léger, Hydrocarbon Processing, March 1985, p. 51-59.

Description of bimetallic catalysts for selective hydrogenations of C₂-, C₃-, C₄-, C₅- and C₅₊-hydrocarbon streams. Particularly bimetallic catalysts of group VIII and group IB metals exhibit improvements in the selectivity compared with pure Pd supported catalysts.

DE-A-2 059 978

Selective hydrogenation of unsaturated hydrocarbons in the liquid phase over a Pd/clay earth catalyst. The catalyst is characterized in that the clay earth support with a BET of 120 m²/g is firstly subjected to a steam treatment at 110-300° C. and is then calcined at 500-1200° C. Finally, the Pd compound is applied and calcined at 300-600° C.

EP-A-0 564 328 and EP-A-0 564 329

Catalyst which consists inter alia of Pd and In or Ga on a support. The catalyst combination permits a use without the addition of CO with high activity and selectivity.

EP-A-0 089 252

Pd, Au supported catalysts.

The catalyst manufacture includes the following steps:
impregnation of a mineral support with a Pd compound
calcination under O₂-containing gas
treatment with a reducing agent
impregnation with a halogenated Au compound
treatment with a reducing agent
washing out the halogen using a basic compound
calcination under O₂-containing gas.

U.S. Pat. No. 5,475,173

Catalyst consisting of Pd and Ag and alkali metal fluoride on inorganic support.

Advantages of the catalyst: As a result of the addition of KF, increased butadiene conversion and better selectivity to butenes (i.e. lower over-hydrogenation to n-butane).

EP-A-O 653 243

Catalyst is characterized in that the active component is found predominantly in the mesopores and macropores. The catalyst is further characterized by a large pore volume and low packing density. For example, the catalyst from example 1 has a packing density of 383 g/l and a pore volume of 1.17 ml/g.

EP-A-0 211 381

Catalyst of group VIII metal (preferably Pt) and at least one metal from Pb, Sn or Zn on an inorganic support. The preferred catalyst consists of Pt/ZnAl₂O₄. Said promoters Pb, Sn and Zn improve the selectivity of the Pt catalyst.

EP-A-0 722 776

Catalyst of Pd and at least one alkali metal fluoride and optionally Ag on an inorganic support (Al₂O₃, TiO₂ and/or ZrO₂). The catalyst combination permits a selective hydrogenation in the presence of sulfur compounds.

EP-A-0 576 828

Catalyst based on noble metal and/or noble metal oxide on Al₂O₃ support with a defined X-ray diffraction pattern. The support consists here of α-Al₂O₃ and/or γ-Al₂O₃. Due to the special support, the catalyst has a high initial selectivity and can therefore be used immediately for the selective hydrogenation of unsaturated compounds.

JP 01110594

Pd supported catalyst

Additionally, a further electron donor is used. This consists either of a metal deposited on the catalyst, such as, for example, Na, K, Ag, Cu, Ga, In, Cr, Mo or La, or an additive to the hydrocarbon feedstock, such as, for example, alcohol, ether or N-containing compounds. Said measures can achieve a reduction in the 1-butene isomerization.

DE-A-31 19 850

Catalyst of an SiO₂ or Al₂O₃ support with 10 to 200 m²/g or ≦100 m 2/g of Pd and Ag as active component. The catalyst serves primarily for the hydrogenation of hydrocarbon streams with a low content of butadiene.

EP-A-0 780 155

Catalyst of Pd and a group IB metal on an Al₂O₃ support, where at least 80% of the Pd and 80% of the group IB metal are applied in an external coating between r₁ (=radius of the pellet) and 0.8-r₁.

Alternative: Extraction of Butadiene from Crude C₄ Cut

The preferred process of isolating butadiene is based on the physical principle of extractive distillation. The addition of selective organic solvents lowers the volatility of specific components of a mixture, in this case butadiene. For this reason, these remain with the solvent in the bottom of the distillation column, while the accompanying substances which could previously not be separated off by distillation can be removed overhead. Solvents used for the extractive distillation are mainly acetone, furfural, acetonitrile, dimethylacetamide, dimethylformamide (DMF) and N-methylpyrrolidone (NMP). Extractive distillations are particularly suitable for butadiene-rich C₄ cracker cuts having a relatively high proportion of alkynes, including methylacetylene, ethylacetylene and vinylacetylene, and methylallene.

The simplified principle of solvent extraction from crude C₄ cut can be described as follows: The completely vaporized C₄ cut is fed to an extraction column at its lower end. The solvent (DMF, NMP) flows from the top in the opposite direction to the gas mixture and on its way down becomes laden with the more soluble butadiene and small amounts of butenes. At the lower end of the extraction column, part of the pure butadiene which has been obtained is fed in in order to drive out the butenes as far as possible. The butenes leave the separation column at the top. In a further column, referred to as a degasser, the butadiene is freed from the solvent by boiling out and is subsequently purified by distillation.

The reaction exit stream from a butadiene extractive distillation is usually fed to the second stage of a selective hydrogenation in order to reduce the residual butadiene content to values of <10 ppm.

The C₄ stream which remains after butadiene has been separated off is referred to as C₄ raffinate or raffinate I and comprises mainly the components isobutene, 1-butene, 2-butenes, and n- and isobutanes.

Separating Off Isobutene from Raffinate I

In the further separation of the C₄ stream, isobutene is preferably isolated subsequently since it differs from the other C₄ components by virtue of its branching and its higher reactivity. Apart from the possibility of a shape-selective molecular sieve separation, by means of which isobutene can be isolated in a purity of 99% and n-butenes and butane adsorbed on the molecular sieve pores can be desorbed again using a higher-boiling hydrocarbon, this is carried out primarily by distillation using a deisobutenizer, by means of which isobutene is separated off together with 1-butene and isobutene at the top, and 2-butenes and n-butane together with residual amounts of iso- and 1-butene remain in the bottoms, or extractively by reaction of isobutene with alcohols over acidic ion exchangers. Methanol (MTBE) or isobutanol (IBTBE) are preferably used for this purpose.

The preparation of MTBE from methanol and isobutene is carried out at 30 to 100° C. and at a pressure slightly above atmospheric pressure in the liquid phase over acidic ion exchangers. The process is carried out either in two reactors or in a two-stage shaft reactor in order to achieve virtually complete isobutene conversion (>99%). The pressure-dependent azeotrope formation between methanol and MTBE requires a multistage pressure distillation to isolate pure MTBE, or is achieved by relatively new technology using methanol adsorption on adsorber resins. All other components of the C₄ fraction remain unchanged. Since small proportions of diolefins and acetylenes can shorten the life of the ion exchanger as a result of polymer formation, preference is given to using bifunctional PD-containing ion exchangers, in the case of which, in the presence of small amounts of hydrogen, only diolefins and acetylenes are hydrogenated. The etherification of the isobutene remains uninfluenced by this.

MTBE serves primarily to increase the octane number of gasoline. MTBE and IBTBE can alternatively be back-cleaved in the gas phase at 150 to 300° C. over acidic oxides to obtain pure isobutene.

A further possibility for separating off isobutene from raffinate I consists in the direct synthesis of oligo/polyisobutene. In this way it is possible, over acidic homogeneous and heterogeneous catalysts, such as e.g. tungsten trioxide and titanium dioxide, and at isobutene conversions up to 95%, to obtain an exit stream which has a residual isobutene content of a maximum of 5%.

Feed Purification of the Raffinate II Stream Over Adsorber Materials

To improve the service life of catalysts used for the subsequent metathesis step, it is necessary, as described above, to use a feed purification (guard bed) for removing catalyst poisons, such as, for example, water, oxygenates, sulfur or sulfur compounds or organic halides.

Processes for adsorption or adsorptive purification are described, for example, in W. Kast, Adsorption aus der Gasphase [Adsorption from the gas phase], VCH, Weinheim (1988). The use of zeolitic adsorbents is described in D. W. Breck, Zeolite Molecular Sieves, Wiley, New York (1974).

The removal of, specifically, acetaldehyde from C₃— to C₁₋₅-hydrocarbons in the liquid phase can be carried out according to EP-A-0 582 901.

Selective Hydrogenation of Crude C₄ Cut

Butadiene (1,2- and 1,3-butadiene) and alkynes or alkenynes present in the C₄ fraction from the crude C₄ fraction originating from a steam cracker or a refinery are firstly selectively hydrogenated in a two-stage process. According to one embodiment, the C₄ stream originating from the refinery can also be fed directly to the second step of the selective hydrogenation.

The first step of the hydrogenation is preferably carried out over a catalyst which comprises 0.1 to 0.5% by weight of palladium on aluminum oxide as support. The reaction is carried out in the gas/liquid phase in a fixed bed (downflow mode) with a liquid cycle. The hydrogenation is carried out at a temperature in the range from 40 to 80° C. and at a pressure of from 10 to 30 bar, a molar ratio of hydrogen to butadiene of from 10 to 50 and an LHSV of up to 15 m³ of fresh feed per m³ of catalyst per hour and a ratio of recycle to feed stream of from 5 to 20.

The second step of the hydrogenation is preferably carried out over a catalyst which comprises 0.1 to 0.5% by weight of palladium on aluminum oxide as support. The reaction is carried out in the gas/liquid phase over a fixed bed (downflow mode) with a liquid cycle. The hydrogenation is carried out at a temperature in the range from 50 to 90° C. and at a pressure from 10 to 30 bar, a molar ratio of hydrogen to butadiene of from 1.0 to 10 and an LHSV of from 5 to 20 m³ of fresh feed per m³ of catalyst per hour and a ratio of recycle to feed stream of from 0 to 15.

The hydrogenation is carried out under “low isom” conditions, under which no or at least the smallest possible C═C isomerization of 1-butene to 2-butene results. The residual content of butadiene can be 0 to 50 ppm depending on the hydrogenation severity.

The reaction exit stream obtained in this way is referred to as raffinate I and, in addition to isobutene, has 1-butene and 2-butene in varying molar ratios.

Alternative: Separating Off Butadiene from Crude C₄ Cut via Extraction

The extraction of butadiene from crude C₄ cut is carried out in accordance with BASF technology using N-methylpyrrolidone.

According to one embodiment of the invention, the reaction exit stream from the extraction is fed to the second stage of the selective hydrogenation described above in order to remove residual amounts of butadiene, during which it must be ensured that no or only slight isomerization of 1-butene to 2-butene results.

Separating Off Isobutene by Means of Etherification with Alcohols

In the etherification stage, isobutene is reacted with alcohols, preferably with isobutanol, over an acidic catalyst, preferably over an acidic ion exchanger, to give ethers, preferably isobutyl tert-butyl ether. According to one embodiment of the invention, the reaction is carried out in a three-stage reactor cascade, in which the reaction mixture flows through flooded fixed-bed catalysts from top to bottom. In the first reactor the inlet temperature is 0 to 60° C., preferably 10 to 50° C.; the outlet temperature is between 25 and 85° C., preferably between 35 and 75° C., and the pressure is 2 to 50 bar, preferably 3 to 20 bar. At a ratio of isobutanol to isobutene of from 0.8 to 2.0, preferably 1.0 to 1.5, the conversion is between 70 and 90%.

In the second reactor the inlet temperature is 0 to 60° C., preferably 10 to 50° C.; the outlet temperature is between 25 and 85, preferably between 35 and 75° C., and the pressure is 2 to 50 bar, preferably 3 to 20 bar. The overall conversion over the two stages increases to 85 to 99%, preferably 90 to 97%.

In the third and largest reactor, equilibrium conversion is achieved at equal inlet and outlet temperatures of from 0 to 60° C., preferably 10 to 50° C. The etherification and removal of the ether formed are followed by ether cleavage: The endothermic reaction is carried out over acidic catalysts, preferably over acidic heterogeneous catalysts, for example phosphoric acid on an SiO₂ support, at an inlet temperature of from 150 to 300° C., preferably at 200 to 250° C., and an outlet temperature of from 100 to 250° C., preferably at 130 to 220° C.

If an FCC C₄ fraction is used, it is to be expected that propane in amounts of around 1% by weight, isobutene in amounts of around 30 to 40% by weight, and C₅-hydrocarbons in amounts of around 3 to 10% will be introduced, which may adversely affect the subsequent process sequence. The work-up of the ether accordingly provides the opportunity of separating off the components mentioned by distillation.

The resulting reaction exit stream, referred to as raffinate II, has a residual isobutene content of from 0.1 to 3% by weight.

If larger amounts of isobutene are present in the exit stream, for example when FCC C₄ fractions are used or when isobutene is separated off by acid-catalyzed polymerization to give polyisobutene (partial conversion), the raffinate stream which remains can, according to one embodiment of the invention, be worked up by distillation prior to further processing.

Purification of the Raffmate II Stream Over Adsorber Materials

The raffinate II stream obtained after the etherification/polymerization (or distillation) is preferably purified over at least one guard bed consisting of high surface-area aluminum oxides, silica gels, aluminosilicates or molecular sieves. The guard bed serves here to dry the C₄ stream and to remove substances which may act as catalyst poisons in the subsequent metathesis step. The preferred adsorber materials are Selexsorb CD and CDO and also 3 Å and NaX molecular sieves (13×). The purification is carried out in drying towers at temperatures and pressures which are chosen such that all components are present in the liquid phase. Optionally, the purification step is used to preheat the feed for the subsequent metathesis step.

The raffinate II stream which remains is virtually free from water, oxygenates, organic chlorides and sulfur compounds.

When the etherification step is carried out with methanol for preparing MTBE, the formation of dimethyl ether as secondary component may make it necessary to combine two or more purification steps or to connect them in series.

Typical compositions of C₄-olefin streams are given below which are obtained in the customary processes mentioned in the description and which, optionally following concentration by distillation or catalytic distillation to the desired 1-butene/2-butene ratio, can be used as starting material stream in the process according to the invention.

Raffinate II:

| Component | % by wt. |
|---|---|
| Isobutane | 5-15 |
| Isobutene | <0.1-5 |
| 1-Butene | 30-60 |
| 1,3-Butadiene | <0-1 |
| n-Butane | 5-25 |
| trans-2-Butene | 10-30 |
| cis-2-Butene | 6-25 |
| Isobutane | 28-52 |
| Isobutene | 26-8 |
| 1-Butene | 6-10 |
| 1,3-Butadiene | 0.1-0.5 |
| n-Butane | 7-13 |
| trans-2-Butene + cis-2-Butene | 31-20 |

Butane Dehydrogenation:

| Component | % by wt. |
|---|---|
| Isobutane | 5-30 |
| cis-2-Butene | 5-30 |
| trans-2-Butene | 5-30 |
| 1,3-Butadiene | 0-5 |
| Propene | 0-5 |
| n-Butanes | Remainder |

Adjustment of the 1-butene/2-butene Ratio

In the event that the C₄ stream to be used according to the invention in the metathesis does not have the desired 1-butene/2-butene ratio, the stream can be enriched with 1-butene by measures known to the person skilled in the art.

A preferred enrichment process is distillation. In principle, the main constituents still present in the C₄ stream can be separated from one another by distillation or fractions can be obtained by distillation which are enriched with one or else two or more components. The boiling points of the main and secondary components at 101.3 kPa are given in the table below.

| Component | Boiling point/° C. |
|---|---|
| Isobutane | −11.7 |
| Isobutene | −6.9 |
| 1-Butene | −6.3 |
| n-Butane | −0.5 |
| trans-2-Butene | +0.9 |
| cis-2-Butene | +3.7 |

If a raffinate II which has the customary composition and comprises the above-mentioned components in varying concentrations is subjected to a distillation, then isobutane is obtained as top product. The middle distillate comprises 1-butene (contaminated with isobutene), while the bottom product mainly has n-butane, trans-2-butene and cis-2-butene. By separating off the top product and removing the middle distillate, it is thus possible to carry out an enrichment of 1-butene. Alternatively, the separation of the C₄ stream and the enrichment with 1-butene can be carried out in two columns.

The enrichment can, on the other hand, preferably be carried out by a combination of catalytic isomerization and distillative separation of the C₄ stream used. As explained above, 1-butene can be separated off from the C₄ mixture by distillation. If the mixture which remains is able to isomerize through the presence of a catalyst, then new 1-butene is constantly formed from 2-butene as a result of the removal of 1-butene from the equilibrium. Such a process can be carried out in two stages, i.e. by carrying out the isomerization and distillation in different processing units. It is also possible to carry out an isomerizing distillation in a single processing unit (catalytic distillation). In this connection, the isomerization catalyst is in the distillation column or in the distillation still. The isomerization catalysts used are known to the person skilled in the art. They generally comprise elements of groups Ia, IIa, IIIb, IVb, Vb or VIII of the Periodic Table of the Elements.

Examples are heterogeneous or homogeneous catalysts comprising RuO₂, MgO, CaO, ZnO, Rb/Cs/K on Al₂O₃, Na on Al₂O₃, K₂CO₃, Na₂CO₃, Pd/Al₂O₃, PdO, boron halides (e.g. BCl₃, BF₃), basic Al₂O₃, oxides of the lanthanide group La₂O₃, Nd₂O₃, NiO mixed catalysts, TiO₂, ZrO₂, C₂O, KC₈, Rb₂O, KF/Al₂O₃, tungstosilicic acid, Co on activated carbon, acidic zeolites, acidic ion exchangers, Co(acac)₂, Fe(CO)₅, Ru²⁺(H₂O)₆ in EtOH, Rh³⁺ complexes.

Catalyst supports for heterogeneous systems which can be used here are, for example, Al₂O₃, SiO₂ or activated carbon.

The fundamental features of the metathesis used in step a) are described, for example, in Ullmann's Encyclopedia of Industrial Chemistry, 5th edition, volume A18, p. 235/236. Further information for carrying out the process can be found, for example, in K. J. Ivin, “Olefin Metathesis”, Academic Press, London, (1983); Houben-Weyl, E18, 1163-1223; R. L. Banks, Discovery and Development of Olefin Disproportionation, CHEMTECH (1986), February, 112-117.

When the metathesis is used on the main constituents 1-butene and 2-butene present in the C₄-olefin streams, in the presence of suitable catalysts, olefins having 5 to 10 carbon atoms, preferably having 5 to 8 carbon atoms, but in particular 2-pentene and 3-hexene, are formed.

Suitable catalysts are preferably molybdenum, tungsten or rhenium compounds. It is particularly expedient to carry out the reaction with heterogeneous catalysis, where the catalytically active metals are used, in particular, in conjunction with supports made of Al₂O₃ or SiO₂. Examples of such catalysts are MoO₃ or WO₃ on SiO₂, or Re₂O₇ on Al₂O₃.

The metathesis reaction is preferably carried out in the presence of heterogeneous metathesis catalysts which have no or only slight isomerization activity and which are chosen from the class of transition metal compounds of metals of the VIb, VIIb or VIII group of the Periodic Table of the Elements applied to inorganic supports.

As metathesis catalyst, preference is given to using rhenium oxide on a support, preferably on γ-aluminum oxide or on Al₂O₃/B₂O₃/SiO₂ mixed supports.

In particular, the catalyst used is Re₂O₇/γ-Al₂O₃ with a rhenium oxide content of from 1 to 20%, preferably 3 to 15%, particularly preferably 6 to 12% % by weight.

The metathesis can be carried out in the liquid phase or preferably in the gas phase.

The metathesis is carried out in the liquid procedure preferably at a temperature of from 0 to 150° C., particularly preferably 20 to 80° C., and a pressure of from 2 to 200 bar, particularly preferably 5 to 30 bar.

If the metathesis is carried out in the gas phase, the temperature is preferably 20 to 300° C., particularly preferably 50 to 200° C. The pressure in this case is preferably 1 to 20 bar, particularly preferably 1 to 5 bar.

It is particularly favorable to carry out the metathesis in the presence of a rhenium catalyst since in this case particularly mild reaction conditions are possible. For example, the metathesis in this case can be carried out at a temperature of from 0 to 50° C. and at low pressures of about 0.1 to 0.2 MPa.

During the dimerization of the olefins or olefin mixtures obtained in the metathesis step, dimerization products are obtained which, with regard to the further processing to give surfactant alcohols, have particularly favorable components and a particularly advantageous composition if a dimerization catalyst is used which contains at least one element from sub-group VIII of the Periodic Table of the Elements, and the catalyst composition and the reaction conditions are chosen such that a dimer mixture is obtained which contains less than 10% by weight of compounds which have a structural element of the formula I (vinylidene group) 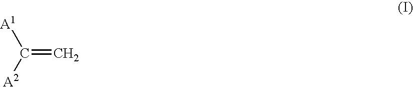
in which A¹ and A² are aliphatic hydrocarbon radicals.

For the dimerization, preference is given to using the internal, linear pentenes and hexenes present in the metathesis product. Particular preference is given to using 3-hexene.

The dimerization may be carried out with homogeneous or heterogeneous catalysis. Preference is given to the heterogeneous procedure since here, firstly, catalyst removal is simplified and the process is thus more economical and, secondly, no environmentally harmful wastewaters are produced, as are usually formed during the removal of dissolved catalysts, for example by hydrolysis. A further advantage of the heterogeneous process is that the dimerization product does not contain halogens, in particular chlorine or fluorine. Homogeneously soluble catalysts generally contain halide-containing ligands, or they are used in combination with halogen-containing cocatalysts. Halogen may be incorporated from such catalyst systems into the dimerization products, which considerably impairs both the product quality and also the further processing, in particular the hydroformylation to give surfactant alcohols.

For heterogeneous catalysis, combinations of oxides of metals from sub-group VIII with aluminum oxide on support materials of silicon oxides and titanium oxides, as are known, for example, from DE-A-43 39 713, are expediently used. The heterogeneous catalyst can be used in a fixed bed—then preferably in coarse form as 1 to 1.5 mm chips—or in suspended form (particle size 0.05 to 0.5 mm). The dimerization is, in the case of the heterogeneous procedure, expediently carried out at temperatures from 80 to 200° C., preferably from 100 to 180° C., under the pressure prevailing at the reaction temperature, optionally also under a protective gas at a pressure above atmospheric, in a closed system. To achieve optimal conversions, the reaction mixture is circulated repeatedly, a certain proportion of the circulating product being continuously discharged and replaced with starting material.

The dimerization according to the invention produces mixtures of monounsaturated hydrocarbons, the components of which predominantly have a chain length twice that of the starting olefins.

Within the scope of the above details, the dimerization catalysts and the reaction conditions are expediently chosen such that at least 80% of the components of the dimerization mixture have, within the region from ¼ to ¾, preferably from ⅓ to ⅔, of the chain length of their main chain, one branch, or two branches on adjacent carbon atoms.

A very characteristic feature of the olefin mixtures prepared according to the invention is their high proportion—generally greater than 75%, in particular greater than 80%—of components with branches, and the low proportion—generally below 25, in particular below 20%—of unbranched olefins. A further characteristic is that predominantly groups having (y-4) and (y-5) carbon atoms are bonded to the branching sites of the main chain, where y is the number of carbon atoms of the monomer used for the dimerization. The value (y-5)=0 means that no side chain is present.

In the case of the C₁₂-olefin mixtures prepared according to the invention, the main chain preferably carries methyl or ethyl groups at the branching points.

The position of the methyl and ethyl groups on the main chain is likewise characteristic: In the case of monosubstitution, the methyl or ethyl groups are in the position P=(n/2)-m of the main chain, where n is the length of the main chain and m is the number of carbon atoms in the side groups, and in the case of disubstitution products, one substituent is in the position P and the other is on the adjacent carbon atom P+1. The proportions of monosubstitution products (single branching) in the olefin mixture prepared according to the invention are characteristically in total in the range from 40 to 75% by weight, and the proportions of double-branched components are in the range from 5 to 25% by weight.

It has also been found that the dimerization mixtures can be further derivatized particularly efficiently when the position of the double bond satisfies certain requirements. In these advantageous olefin mixtures, the position of the double bonds relative to the branches is characterized in that the ratio of the aliphatic hydrogen atoms to olefinic hydrogen atoms is in the range H_{aliph.}:H_{olefin.}=(2*n−0.5): 0.5 to (2*n−1.9): 1.9, where n is the number of carbon atoms of the olefin obtained in the dimerization. (Aliphatic hydrogen atoms is the term used for those which are bonded to carbon atoms which do not participate in a C═C double bond (pi-bond), and olefinic hydrogen atoms is the term used to describe those bonded to the carbon atom which enters into a pi-bond with the adjacent carbon atom.)

Particular preference is given to dimerization mixtures in which the ratio is
H_{aliph.}:H_{olefi.}=(2*n−1.0): 1 to (2*n−1.6): 1.6.

The novel olefin mixtures obtainable by the process according to the invention and having the abovementioned structural features are likewise provided by the present invention. They are valuable intermediates, in particular for the production, described below, of branched primary alcohols and surfactants, but can also be used as starting materials in other industrial processes starting from olefins, particularly when the end products are to have improved biodegradability.

If the olefin mixtures according to the invention are to be used for the production of surfactants, then they are firstly derivatized by processes known per se to give surfactant alcohols.

There are various ways of achieving this, which either involve the direct or indirect addition of water (hydration) to the double bond, or an addition of CO and hydrogen (hydroformylation) to the C═C double bond.

Hydration of the olefins resulting from step c) is expediently carried out by direct water addition with proton catalysis. Of course, this is also possible, for example, via the addition of high-percentage sulfuric acid to give an alkanol sulfonate and subsequent saponification to give the alkanol. The more expedient direct addition of water is carried out in the presence of acidic, in particular heterogeneous, catalysts and usually at a very high olefin partial pressure and at very low temperatures. Suitable catalysts prove to be, in particular, phosphoric acid on supports, such as, for example, SiO₂ or Celite, or else acidic ion exchangers. The choice of conditions depends on the reactivity of the olefins to be reacted and can routinely be ascertained by preliminary experiments (Lit.: e.g. A. J. Kresge et al. J. Am. Chem. Soc. 93, 4907 (1971); Houben-Weyl vol. 5/4 (1960), pages 102-132 and 535-539). Hydration generally leads to mixtures of primary and secondary alkanols in which the secondary alkanols predominate.

For the production of surfactants, it is more favorable to start from primary alkanols. It is therefore preferable to hydroformylate the derivatization of the olefin mixtures obtained from step c) by reaction with carbon monoxide and hydrogen in the presence of suitable, preferably cobalt- or rhodium-containing, catalysts to give branched primary alcohols.

A further preferred subject-matter of the present invention is therefore a process for preparing mixtures of primary alkanols which are suitable, inter alia, for the further processing to give surfactants, by hydroformylation of olefins, which comprises using the above-described olefin mixtures according to the invention as starting material.

A good overview of the process of hydroformylation with numerous further literature references can be found, for example, in the extensive article by Beller et al. in Journal of Molecular Catalysis, A104 (1995) 17-85 or in Ullmann's Encyclopedia of Industrial Chemistry, volume A5 (1986), page 217 et seq., page 333, and the literature references relating to this.

The comprehensive information given therein allows the person skilled in the art to hydroformylate even the olefins produced according to the invention which have a high proportion of branched and internal olefins. In this reaction, CO and hydrogen is added to olefinic double bonds, giving mixtures of aldehydes and alkanols according to the following reaction scheme (in which, for reasons of clarity, the reaction is shown with a linear, terminal olefin): 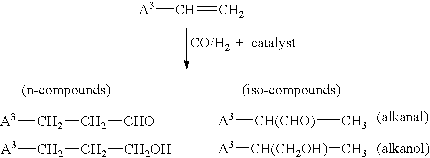
(A³=hydrocarbon radical)

The molar ratio of n and iso compounds in the reaction mixture is usually in the range from 1:1 to 20:1 depending on the hydroformylation process conditions chosen and the catalyst used. The hydroformylation is normally carried out in the temperature range from 90 to 200° and at a CO/H₂ pressure of from 2.5 to 35 MPa (25 to 350 bar). The mixing ratio of carbon monoxide to hydrogen depends on whether the intention is to preferentially produce alkanals or alkanols. The CO:H₂ ratio is advantageously from 10:1 to 1:10, preferably from 3:1 to 1:3, where, for the preparation of alkanals, the range of low hydrogen partial pressures is chosen, and, for the preparation of alkanols, the range of high hydrogen partial pressures is chosen, e.g. CO:H₂=1:2.

Suitable catalysts are mainly metal compounds of the formula HM(CO)₄ or M₂(CO)₈, where M is a metal atom, preferably a cobalt, rhodium or ruthenium atom.

Generally, under hydroformylation conditions, the catalysts or catalyst precursors used in each case form catalytically active species of the formula HₓM_{y}(CO)_{z}L_{q}, in which M is a metal of subgroup VIII, L is a ligand, which can be a phosphine, phosphite, amine, pyridine or any other donor compound, including in polymeric form, and q, x, y and z are integers depending on the valency and type of metal, and the covalence of the ligand L, where q can also be 0.

The metal M is preferably cobalt, ruthenium, rhodium, palladium, platinum, osmium or iridium and in particular cobalt, rhodium or ruthenium.

Suitable rhodium compounds or complexes are, for example, rhodium(II) and rhodium(III) salts, such as rhodium(III) chloride, rhodium(III) nitrate, rhodium(III) sulfate, potassium rhodium sulfate, rhodium(II) or rhodium(III) carboxylate, rhodium(II) and rhodium(III) acetate, rhodium (III) oxide, salts of rhodium(III) acid, such as, for example, trisammonium hexachlororhodate(III). Also suitable are rhodium complexes such as rhodium biscarbonyl acetylacetonate, acetylacetonatobisethylenerhodium(I). Preference is given to using rhodium biscarbonyl acetylacetonate or rhodium acetate.

Suitable cobalt compounds are, for example, cobalt(II) chloride, cobalt(II) sulfate, cobalt(II) carbonate, cobalt(II) nitrate, their amine or hydrate complexes, cobalt carboxylates, such as cobalt acetate, cobalt ethylhexanoate, cobalt naphthenoate, and the cobalt caprolactamate complex. Here, too, it is possible to use the carbonyl complexes of cobalt, such as dicobalt octacarbonyl, tetracobalt dodecacarbonyl and hexacobalt hexadecacarbonyl.

Said compounds of cobalt, rhodium and ruthenium are known in principle and are described adequately in the literature, or they can be prepared by the person skilled in the art in a manner analogous to that for compounds already known.

The hydroformylation can be carried out with the addition of inert solvents or diluents or without such an addition. Suitable inert additives are, for example, acetone, methyl ethyl ketone, cyclohexanone, toluene, xylene, chlorobenzene, methylene chloride, hexane, petroleum ether, acetonitrile, and the high-boiling fractions from the hydroformylation of the dimerization products.

If the resulting hydroformylation product has too high an aldehyde content, this can be removed in a simple manner by a hydrogenation, for example using hydrogen in the presence of Raney nickel or using other catalysts known for hydrogenation reactions, in particular catalysts containing copper, zinc, cobalt, nickel, molybdenum, zirconium or titanium. In the process, the aldehyde fractions are largely hydrogenated to give alkanols. A virtually residue-free removal of aldehyde fractions in the reaction mixture can, if desired, be achieved by posthydrogenation, for example under particularly mild and economical conditions using an alkali metal borohydride.

The mixtures of branched primary alkanols, preparable by hydroformylation of the olefin mixtures according to the invention, are likewise provided by the present invention.

Nonionic or anionic surfactants can be prepared from the alkanols according to the invention in different ways.

Nonionic surfactants are obtained by reacting the alkanols with alkylene oxides of the formula II 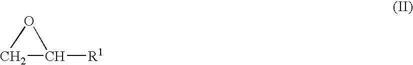
in which R¹ is hydrogen or a straight-chain or branched aliphatic radical of the formula CₙH₂ₙ₊₁, and n is a number from 1 to 16, preferably from 1 to 8. In particular, R¹ is hydrogen, methyl or ethyl.

The alkanols according to the invention can be reacted with a single alkylene oxide species or with two or more different species. The reaction of the alkanols with the alkylene oxides forms compounds which in turn carry an OH group and can therefore react afresh with one molecule of alkylene oxide. Therefore, depending on the molar ratio of alkanol to alkylene oxide, reaction products are obtained which have longer or shorter polyether chains. The polyether chains can contain from 1 to about 200 alkylene oxide structural groups. Preference is given to compounds whose polyether chains contain from 1 to 10 alkylene oxide structural groups.

The chains can consist of identical chain members, or they can have different alkylene oxide structural groups which differ from one another by virtue of their radical R¹. These various structural groups can be present within the chain in random distribution or in the form of blocks.

The reaction equation below serves to illustrate the alkoxylation of the alkanols according to the invention using the example of a reaction with two different alkylene oxides which are used in varying molar amounts x and y.

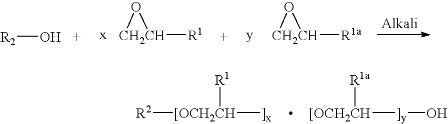

R¹ and R^{1a} are different radicals within the scope of the definitions given for R¹, and R²-OH is a branched alkanol according to the invention.

The alkoxylation is preferably catalyzed by strong bases, which are expediently added in the form of an alkali metal hydroxide or alkaline earth metal hydroxide, usually in an amount of from 0.1 to 1% by weight, based on the amount of the alkanol R²—OH (cf. G. Gee et al., J. Chem. Soc. (1961), p. 1345; B. Wojtech, Makromol. Chem. 66, (1966), p. 180).

Acidic catalysis of the addition reaction is also possible. As well as Brönsted acids, Lewis acids, such as, for example, AlCl₃ or BF₃, are also suitable (cf. P. H. Plesch, The Chemistry of Cationic Polymerization, Pergamon Press, New York (1963)).

The addition reaction is carried out at temperatures of from about 120 to about 220° C., preferably from 140 to 160° C., in a sealed vessel. The alkylene oxide or the mixture of different alkylene oxides is introduced into the mixture of alkanol mixture according to the invention and alkali under the vapor pressure of the alkylene oxide mixture prevailing at the chosen reaction temperature. If desired, the alkylene oxide can be diluted by up to about 30 to 60% using an inert gas. This leads to additional security against explosive polyaddition of the alkylene oxide.

If an alkylene oxide mixture is used, then polyether chains are formed in which the various alkylene oxide building blocks are distributed in a virtually random manner. Variations in the distribution of the building blocks along the polyether chain arise due to varying reaction rates of the components and can also be achieved arbitrarily by continuous introduction of an alkylene oxide mixture of a program-controlled composition. If the various alkylene oxides are reacted successively, then polyether chains having block-like distribution of the alkylene oxide building blocks are obtained.

The length of the polyether chains varies within the reaction product in a random manner about a mean, essentially the stoichiometric value arising from the amount added.

The alkoxylates preparable starting from alkanol mixtures and olefin mixtures according to the invention are likewise provided by the present invention. They exhibit very good surface activity and can therefore be used as neutral surfactants in many areas of application.

Starting from the alkanol mixtures according to the invention, it is also possible to prepare surface-active glycosides and polyglycosides (oligoglycosides). These substances too have very good surfactant properties. They are obtained by single or multiple reaction (glycosidation, polyglycosidation) of the alkanol mixtures according to the invention with mono-, di- or polysaccharides with the exclusion of water and with acid catalysis. Suitable acids are, for example, HCl or H₂SO₄. As a rule, the process produces oligoglycosides having random chain length distribution, the average degree of oligomerization being from 1 to 3 saccharide residues.

In another standard synthesis, the saccharide is firstly acetalized under acid catalysis with a low molecular weight alkanol, e.g. butanol, to give butanol glycoside. This reaction can also be carried out with aqueous solutions of the saccharide. The lower alkanol glycoside, for example butanol glycoside, is then reacted with the alkanol mixtures according to the invention to give the desired glycosides according to the invention. After the acidic catalyst has been neutralized, excess long-chain and short-chain alkanols can be removed from the equilibrium mixture, e.g. by distillating off under reduced pressure.

Another standard method proceeds via the O-acetyl compounds of saccharides. The latter are converted, using hydrogen halide preferably dissolved in glacial acetic acid, into the corresponding O-acetylhalosaccharides, which react in the presence of acid-binding agents with the alkanols to give the acetylated glycosides.

Preferred for the glycosidation of the alkanol mixtures according to the invention are monosaccharides, either hexoses, such as glucose, fructose, galactose, mannose, or pentoses, such as arabinose, xylose or ribose. Particular preference for glycosidation of the alkanol mixtures according to the invention is glucose. It is, of course, also possible to use mixtures of said saccharides for the glycosidation. Glycosides having randomly distributed sugar residues are obtained, depending on the reaction conditions. The glycosidation can also take place several times, resulting in polyglycoside chains being added to the hydroxyl groups of the alkanols. In a polyglycosidation using different saccharides, the saccharide building blocks can be randomly distributed within the chain or form blocks of the same structural groups.

Depending on the reaction temperature chosen, furanose or pyranose structures can be obtained. To improve the solubility ratios, the reaction can also be carried out in suitable solvents or diluents.

Standard processes and suitable reaction conditions have been described in various publications, for example in “Ullmann's Encyclopedia of Industrial Chemistry”, 5th edition vol. A25 (1994), pages 792-793 and in the literature references given therein, by K. Igarashi, Adv. Carbohydr. Chem. Biochem. 34, (1977), pp. 243-283, by Wulff and Röhle, Angew. Chem. 86, (1974), pp. 173-187, or in Krauch and Kunz, Reaktionen der organischen Chemie [Reactions in Organic Chemistry], pp. 405-408, Hüthig, Heidelberg, (1976).

The glycosides and polyglycosides (oligoglycosides) preparable starting from alkanol mixtures and olefin mixtures according to the invention are likewise provided by the present invention.

Both the alkanol mixtures according to the invention and the polyethers prepared therefrom can be converted into anionic surfactants by esterifying (sulfating) them in a manner known per se with sulfuric acid or sulfuric acid derivatives to give acidic alkyl sulfates or alkyl ether sulfates, or with phosphoric acid or its derivatives to give acidic alkyl phosphates or alkyl ether phosphates.

Sulfating reactions of alcohols have already been described, e.g. in U.S. Pat. No. 3,462,525, 3,420,875 or 3,524,864. Details on carrying out this reaction can also be found in “Ullmann's Encyclopedia of Industrial Chemistry”, 5th edition vol. A25 (1994), pages 779-783 and in the literature references given therein.

If sulfuric acid itself is used for the esterification, then from 75 to 100% strength by weight, preferably from 85 to 98% strength by weight, of acid is advantageously used (“concentrated sulfuric acid” or “monohydrate”). The esterification can be carried out in a solvent or diluent if one is desired for controlling the reaction, e.g. the evolution of heat. In general, the alcoholic reactant is initially introduced, and the sulfating agent is gradually added with continuous mixing. If complete esterification of the alcohol component is desired, the sulfating agent and the alkanol are used in a molar ratio from 1:1 to 1:1.5, preferably from 1:1 to 1:1.2. Lesser amounts of sulfating agent can be advantageous if mixtures of alkanol alkoxylates according to the invention are used and the intention is to prepare combinations of neutral and anionic surfactants. The esterification is normally carried out at temperatures from room temperature to 85° C., preferably in the range from 45 to 75° C.

In some instances, it may be advantageous to carry out the esterification in a low-boiling water-immiscible solvent and diluent at its boiling point, the water forming during the esterification being distilled off azeotropically.

Instead of sulfuric acid of the concentration given above, for the sulfation of the alkanol mixtures according to the invention it is also possible, for example, to use sulfur trioxide, sulfur trioxide complexes, solutions of sulfur trioxide in sulfuric acid (“oleum”), chlorosulfonic acid, sulfuryl chloride and also amidosulfonic acid. The reaction conditions should then be adapted appropriately.

If sulfur trioxide is used as sulfating agent, then the reaction can also be carried out advantageously in a falling-film reactor in countercurrent, if desired also continuously.

Following esterification, the mixtures are neutralized by adding alkali and, optionally after removal of excess alkali metal sulfate and any solvent present, are worked up.

The acidic alkanol sulfates and alkanol ether sulfates and salts thereof obtained by sulfation of alkanols and alkanol ethers according to the invention and their mixtures are likewise provided by the present invention.

In an analogous manner, alkanols and alkanol ethers according to the invention and mixtures thereof can also be reacted with phosphating agents (phosphated) to give acidic phosphoric esters.

Suitable phosphating agents are mainly phosphoric acid, polyphosphoric acid and phosphorus pentoxide, but also POCl₃ when the remaining acid chloride functions are subsequently hydrolyzed. The phosphation of alcohols has been described, for example, in Synthesis 1985, pages 449 to 488.

The acidic alkanol phosphates and alkanol ether phosphates obtained by phosphation of alkanols and alkanol ethers according to the invention and their mixtures are also provided by the present invention.

Finally, the use of the alkanol ether mixtures, alkanol glycosides and the acidic sulfates and phosphates of the alkanol mixtures and of the alkanol ether mixtures preparable starting from the olefin mixtures according to the invention as surfactants is also provided by the present invention.

The working examples below illustrate the production and use of the surfactants according to the invention.

### EXAMPLES 1 TO 6

### Metathesis of C4-Mixtures

Olefin streams which have been produced by the processes mentioned in the description are, if necessary, brought to the given 1-butene/2-butene ratio by catalytic or noncatalytic distillation. If necessary, isobutene present is removed by literature-known processes to a residual content of <3% by etherification.

The C4-olefin stream with the composition given in the table below is firstly passed over a 13× molecular sieve in order to remove oxygenates, compressed to the reaction pressure of 40 bar, mixed, in the given ratio, with freshly added ethene (measurement by weighing the difference), and the appropriate C4 recycle stream is set. The C4 recycle stream is chosen here such that a total butene conversion of 75% is achieved. Moreover, amounts of C4 produced are removed from the system in order to prevent accumulation of butanes (so-called C4 purge). The C5 recycle stream separated off in the 2nd column is recycled completely upstream of the reactor in order to suppress the cross-metathesis between 1-butene and 2-butene. The reaction mixture is metathesized in a 500 ml tubular reactor using a 10% strength Re₂O₇ catalyst. The temperature is 40° C.

The exit stream is separated into a C2/3, C4, C5 and C6 stream using three columns, and the individual streams are analyzed by gas chromatography.

The balances were established in each case for 24 h at a constant reaction temperature.

TABLE
Metathesis of various C4-olefin streams

| | | n-Butene | | | | | | | |
|---|---|---|---|---|---|---|---|---|---|
| | | content in | 1-Butene/ | | | | Ethene | 3-Hexene | Propene |
| | Source of | olefin stream | 2-butene | 1-Butene | 2-Butene | C4 feed | addition | yield | yield |
| Example | C4-olefin stream | [%] | ratio | g/h | g/h | [g/h] | [g/h] | [g/h] | [g/h] |
| 1 | Raffinate II | 90 | 2 | 600 | 300 | 1000 | 0 | 320 | 320 |
| 2 | C4-olefin stream ex | 31 | 1.2 | 170 | 140 | 1000 | 21 | 100 | 160 |
| | dehydrogenation | | | | | | | | |
| 3 | Dehydrogenation + distillation | 56 | 1.4 | 330 | 230 | 1000 | 26 | 180 | 260 |
| 4 | Dehydrogenation + catalytic | 57 | 1.8 | 370 | 200 | 1000 | 7 | 210 | 230 |
| | (isomerizing) | | | | | | | | |
| | distillation | | | | | | | | |
| 5 | FCC process | 41 | 0.4 | 140 | 270 | 1000 | 83 | 70 | 320 |
| (comparison) | | | | | | | | | |
| 6 | FCC + catalytic | 71 | 1.9 | 470 | 240 | 1000 | 5 | 260 | 280 |
| | distillation | | | | | | | | |

Dimerization of the Resulting Olefins

### EXAMPLE 7

704 g of 3-hexene prepared by metathesis of a C4-olefin stream ex FCC+catalytic distillation as in example 6 are passed at a flow rate of 37 g/h at 60° C. into a tubular reactor which contains 793 g of an NiO/SiO2/TiO2 mixed catalyst. The discharge is separated by means of a packed column by distillation into C6 and high-boiling components (C12+), unreacted hexene is recycled to the reactor (102 g/h, conversion in a straight pass about 27%). The high-boiling component discharge is then separated by distillation into its constituents.

After 10 h, the experiment is complete. Yield: 494 g of C12, 122 g of C18, 40 g of C24. Differences arise as a result of the remaining hold-up in the reactor.

The resulting isomer mixture dodecene can be used in the hydroformylation (see examples 13).

Examples 8-12 were carried out analogously to example 7:

### EXAMPLES 8-12

| | | Amount | | High- boiling |
|---|---|---|---|---|
| | | of | Dodecene | component |
| | | 3-hexene | selectivity | selectivity |
| Example | Source of C4 stream | [g] | [%] | [%] |
| 8 | Raffinate II (Ex. 1) | 1 220 | 76 | 24 |
| 9 | Raffinate II (Ex. 1) | 1 621 | 78 | 22 |
| 10 | Dehydrogenation + | 2 113 | 81 | 19 |
| | catalytic | | | |
| | distillation (Ex. 4) | | | |
| 11 | FCC + catalytic | 1 160 | 79 | 21 |
| | distillation (Ex. 6) | | | |
| 12 | C4-olefin stream ex | 1 780 | 82 | 18 |
| | dehydrogenation | | | |
| | (Ex. 2) | | | |

An exemplary skeletal isomer composition of the isomer mixture obtained from example is given below:
14.3% of n-dodecene
32.2% of methylundecenes
30.2% of ethyldecenes
5.5% of dimethyldecenes
9.7% of ethylmethylnonenes
3.7% of diethylnonenes

### EXAMPLE 13

### Hydroformylation of the Dodecene Prepared by Metathesis of Various C4-Olefln Streams and Dimerization of the Hexene Resulting Therefrom

In a 2.5 l autoclave with lifter stirrer, 1 050 g of dodecene (prepared as in example 12) are hydroformylated using 4.4 g of CO₂(CO)₈ with the addition of 110 g of water at 185° C. and 280 bar of a CO/H₂ gas mixture (1/1) for 7.5 hours. The autoclave is cooled, decompressed and emptied. The reaction product is stirred with 500 ml of 10% strength acetic acid and with the introduction of air at 80° C. for 30 minutes. The aqueous phase is separated off and the organic phase is washed with 2×1 l of water. This gives 1 256 g of product (conversion: 93%).

Hydrogenation with Raney Ni:

2 460 g of an oxo product prepared in this way are hydrogenated in a 5 l autoclave with lifter stirrer with the addition of 10% by weight of water with 100 g of Raney nickel at 150° C. and a hydrogen pressure of 280 bar for 15 hours. The system is then left to reach room temperature and decompress and the product is filtered off with suction over kieselguhr. Fractional distillation gives 1 947 g of a tridecanol mixture.

After-Hydrogenation with NaBH₄:

1 947 g of such a tridecanol mixture are stirred under an argon atmosphere with 15 g of NaBH₄ at 80° C. for 18 hours. The mixture is left to cool to 50° C., 500 g of dilute sulfuric acid are slowly added dropwise and the mixture is stirred for another 30 minutes. After the aqueous phase has been separated off, 500 g of sodium hydrogencarbonate solution are added dropwise and the mixture is stirred for 30 minutes. The aqueous phase is separated off, the organic phase is washed with 2×500 ml of water and fractionally distilled. This gives 1 827 g of a tridecanol which has an OH number of 277 mg of KOH/g.

### EXAMPLE 14

### Hydroformylation of the Dodecene Prepared by Metathesis of Various C4-Olefin Streams and Dimerization of the Hexene Obtained Therefrom

In a 2.5 l autoclave with lifter stirrer, 1 072 g of dodecene are hydroformylated using 4.5 g of CO₂ (CO)₈ with the addition of 110 g of water at 185° C. and 280 bar of a CO/H₂ gas mixture (1/1) for 7.5 hours. The autoclave is cooled, decompressed and emptied. The reaction product is stirred with 500 ml of 10% strength acetic acid and with the introduction of air at 80° C. for 30 minutes. The aqueous phase is separated off and the organic phase is washed with 2×1 l of water. This gives 1 268 g of product (conversion: 92%).

Hydrogenation with Raney Ni:

2 422 g of an oxo product prepared in this way are hydrogenated in a 5 l autoclave with lifter stirrer with the addition of 10% by weight of water with 100 g of Raney nickel at 150° C. and a hydrogen pressure of 280 bar for 15 hours. The system is then left to reach room temperature and decompress and the product is filtered off with suction over kieselguhr. Fractional distillation gives 1 873 g of a tridecanol mixture.

After-Hydrogenation with NaBH₄:

1 873 g of such a tridecanol mixture are stirred under an argon atmosphere with 15 g of NaBH₄ at 80° C. for 18 hours. The mixture is left to cool to 50° C., 500 g of dilute sulfuric acid are slowly added dropwise and the mixture is stirred for another 30 minutes. After the aqueous phase has been separated off, 500 g of sodium hydrogencarbonate solution are added dropwise and the mixture is stirred for 30 minutes. The aqueous phase is separated off, the organic phase is washed with 2×500 ml of water and fractionally distilled. The after-hydrogenation is repeated with 10 g of NaBH₄ in the manner described. This gives 1 869 g of a tridecanol which has an OH number of 278 mg of KOH/g. The average degree of branching was determined by means of 1H-NMR spectroscopy via the methyl group signals as 1.5.

### EXAMPLE 15

### Hydroformylation of the Dodecene Prepared by Metathesis of Various C4-Olefin Streams and Dimerization of the Hexene Obtained Therefrom

The dodecene is hydroformylated continuously in two cascaded autoclaves with lifter stirrers, using 15 ppm of rhodium biscarbonylacetylacetonate and 210 ppm of a polyethyleneimine in which 60% of all nitrogen atoms have been acylated with lauric acid. The hydroformylation was carried out at 150° C. and a synthesis gas pressure (CO/H₂=1/1) of 280 bar with an average residence time of 5.3 hours. The reaction product was separated in a wiper-blade evaporator at 170° C. and 20 mbar.

The oxo products prepared were subjected to a fixed-bed hydrogenation in trickle mode using a Co/Mo fixed-bed catalyst. The reaction was carried out at 170° C. and 280 bar of hydrogen with the addition of 10% by weight of water with a space velocity of 0.1 kg/1 h.

Following distillative work-up, the tridecanol mixture was after-hydrogenated with NaBH4 and finally dried over molecular sieve. The average oxo yield was 86%.

The OH number of the prepared tridecanol is 278 mg of KOH/g.

1. A process for preparing surfactant alcohols and surfactant alcohol ethers by derivatization of olefins having about 10 to 20 carbon atoms or of mixtures of such olefins and optional subsequent alkoxylation which comprises
(a) subjecting a C₄-olefin mixture with a 1-butene/2-butene ration of ≧1.2 to a metathesis,
(b) separating off olefins having 5 to 8 carbon atoms from the metathesis mixture,
(c) subjecting the separated-off olefins individually or in a mixture to a dimerization to give olefin mixtures having 10 to 16 carbon atoms,
(d) subjecting the resulting olefin mixture, optionally after a fractionation, to derivatization to give a mixture of surfactant alcohols and
(e) optionally alkoxylating the surfactant alcohols.

2. A process as claimed in claim 1, wherein the 1-butene/2-butene ratio has values of ≧1.4.

3. A process as claimed in claim 1, which comprises adjusting the desired 1-butene/2-butene ratio of the C₄-olefin stream prior to use in the metathesis by distillation or by distillation combined with an isomerization.

4. A process as claimed in claim 1, which comprises obtaining the C₄-olefin mixture by cracking higher hydrocarbons, optionally followed by the removal of undesired by-products.

5. A process as claimed in claim 4, wherein the cracking takes place by FCC cracking or by steam cracking.

6. A process as claimed in claim 1, wherein the C₄-olefin mixture is obtained from LNG, LPG or MTO streams.

7. A process as claimed in claim 6, wherein the olefin mixture is obtained by dehydrogenating of the C₄ fraction in the LPG stream and optional removal of dienes, alkynes and enynes formed.

8. A process as claimed in claim 6, wherein the olefin mixture is obtained from an LNG stream which is converted into the desired olefins via a MTO process.

9. A process as claimed in claim 1, wherein step a), the metathesis is carried out in the presence of molybdenum-, tungsten- or rhenium-containing catalysts.

10. A process as claimed in claim 1, wherein, in step b), the olefins having 5 and 6 carbon atoms are separated off.

11. A process as claimed in claim 1, wherein step c), the dimerization, is carried out under heterogeneous catalysis.

12. A process as claimed in claim 1, wherein a dimerization catalyst is used which contains at least one element from sub-group VIII of the Periodic Table of the Elements, and the catalyst composition and the reaction conditions are chosen such that a dimer mixture is obtained which comprises less than 10% by weight of compounds which have a structural element of the formula I (vinylidene group) 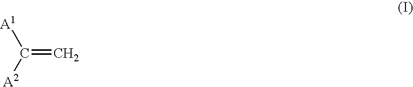
in which A¹ and A² are aliphatic hydrocarbon radicals.

13. A process as claimed in claim 1, wherein, in step c), the olefins having 5 and 6 carbon atoms are dimerized individually or in a mixture.

14. A process as claimed in claim 1, wherein, in step c), the 3-hexene is dimerized.

15. A process as claimed in claim 1, wherein the derivatization (step d)) is carried out by hydroformylation.

16. A novel olefin mixture which can be prepared by steps 1), b) and c) of the process of claim 1.

17. An olefin mixture as claimed in claim 16, which has a proportion of unbranched olefins of less than 25% by weight.

18. An olefin mixture as claimed in claim 16, wherein at lest 80% of the components of the dimerization mixture have, within the region from ¼ to ¾, preferably from ⅓ to ⅔, of the chain length of their main chain, one branch or two branches on adjacent carbon atoms.

19. An olefin mixture as claimed in claim 16, wherein predominantly groups having (y-4) and (y-5) carbon atoms are bonded to the branching sites of the main chain, where y is the number of carbon atoms of the monomer used for the dimerization.

20. An olefin mixture as claimed in claim 16, wherein the ratio of the aliphatic to olefinic hydrogen atoms is in the range H_{aliph.}:H_{olefin.}=(2*n-0.5): 1 to (2*n-1.9): 1.9, where n is the number of carbon atoms of the olefin obtained in the dimerization.

21. An olefin mixture as claimed in claim 16, wherein the ratio of the aliphatic to olefinic hydrogen atoms is in the range H_{aliph.}:H_{olefin.}=(2*n-1.0): 1 to (2*n-1.6): 1.6.

22. A novel surfactant alcohol which can be prepared by steps a), b), c), d) and optionally e) of the process as claimed in claim 1, and an alkoxylation product thereof.

23. A nonionic surfactant comprising the surfactant alcohol alkoxylation products as claimed in claim 22.

24. (canceled)

25. A process for producing alkanol glycoside and polyglycoside mixtures said process comprising single or multiple reaction of the surfactant alcohols as claimed in claim 22 with mono-, di or polysaccharides with the exclusion of water under acid catalysis or with O-acetylsaccharide halides.

26. A process for producing surface-active sulfates said process comprising esterification of the surfactant alcohols and their alkoxylation products as claimed in claim 22 with sulfuric acid or sulfuric acid derivatives to give acidic alkyl sulfates or alkyl ether sulfates.

27. A process for producing surface-active phosphates said process comprising esterification of the surfactant alcohols and their alkoxylation products as claimed in claim 22 with phosphoric acid or derivatives threof to give acidic alkyl phosphates or alkyl ether phosphates.
